(19) United States
(12) Patent Application Publication
(10) Pub. No.: US 2008/0139969 A1
(43) Pub. Date: Jun. 12, 2008

(54) Fabric Sensor and a Garmet Incorporating the Sensor

(75) Inventors: Paul A. Gough, Smallfield (GB); Matthew J. Bickerton, Redhill (GB)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/571,097

(22) PCT Filed: Jun. 29, 2005
(86) PCT No.: PCT/IB05/52165

§ 371 Date: Jan. 24, 2008

(30) Foreign Application Priority Data
Jul. 1, 2004 (GB) 0414731.0

(51) Int. Cl. A61B 5/103; A41D 1/00

(52) U.S. Cl. 600/595; 2/69

(57) ABSTRACT

A fabric sensor for measuring body movement comprises first and second gripping sections and a third sensing section intermediate of the first and second sections. The third sensing section has a lower amount of grip than either the first or second gripping sections. The third sensing section comprises a construction of first and second fibres, the first fibres being of a conductive nature, and the second fibres being of an elastic nature. A garment comprising one or more substantially tubular sections for receiving a body portion and including one or more of the fabric sensors is also described.

This invention relates to a fabric sensor, a method of forming a fabric sensor, a garment incorporating the sensor and a method of forming a garment.

It is well known to place sensors in garments. These sensors are to measure physiological parameters of the wearer and to provide the measured data for further use. Typically such garments are used in medical and sports environments to measure the performance of a patient or athlete in defined circumstances, for the purpose of evaluating their physiological performance. They are also used in motion capture work for the film and game industries.

One such garment is disclosed in International patent application publication WO 03/095020, which discloses a garment that is provided with a sensor band of generally smaller dimensions than the garment for holding sensor electrodes incorporated in the band against a users body while wearing the garment. The sensor band is elasticated to conform against the users body and the garment is relatively loose fitting. The sensor band is attached to the remainder of the garment by highly elastic and flexible webbing portions. The sensor used is a heart rate monitor (HRM). The garment holds the HRM tightly against the body to ensure a good measurement can be achieved.

A more complicated arrangement is disclosed in United States of America patent publication U.S. Pat. No. 6,050,962, which discloses a sensing system that is provided for measuring various joints of a human body for applications for performance animation, biomechanical studies and general motion capture. One sensing device of the system is a linkage-based sensing structure comprising rigid links interconnected by revolute joints, where each joint angle is measured by a resistive bend sensor or other convenient goniometer. Such a linkage-based sensing structure is typically used for measuring joints of the body, such as the shoulders, hips, neck, back and forearm, which have more than a single rotary degree of freedom of movement. In one embodiment of the linkage-based sensing structure, a single long resistive bend sensor measures the angle of more than one revolute joint. The terminal ends of the linkage-based sensing structure are secured to the body such that movement of the joint is measured by the device. A second sensing device of the sensing system comprises a flat, flexible resistive bend sensor guided by a channel on an elastic garment. Such a flat sensing device is typically used to measure various other joints of the body which have primarily one degree of freedom of movement, such as the elbows, knees and ankles. Combining the two sensing devices as described, the sensing system has low sensor bulk at body extremities, yet accurately measures the multi-degree-of-freedom joints nearer the torso. Such a system can operate totally untethered, in real time, and without concern for electromagnetic interference or sensor occlusion.

The system described in this patent has two types of sensor. The first is non-fabric sensor and comprises a system of rigid links. This type of sensor is inappropriate in the vast majority of applications, because the presence of the sensors on the body of the user will affect the performance. For example, if the serve of a tennis player is being measured, then a system of rigid links will make it impossible for the player to execute their serve as they would normally. The second type of sensor is a resistive bend sensor, placed in a channel or pocket of the elastic garment. This type of sensor is similar to that disclosed in the first mentioned patent application, being a sensor held tightly against the body of the user.

However, when measuring the change in position of a user's joint using a stretch sensor held against the user's body, a significant problem can occur. This is caused by the fact that when a user is moving their body, a garment will move relative to the body, causing unwanted artefacts to occur in sensors, where, in fact, no movement of the joint has occurred. This causes erroneous results to be returned by sensors, with the result that unreliable data is then used.

It is therefore an object of the invention, to improve upon the known art.

According to a first aspect of the present invention, there is provided a fabric sensor for measuring body movement comprising first and second gripping sections and a third sensing section intermediate of the first and second sections, the third sensing section having a lower amount of grip than either the first or second gripping sections.

According to a second aspect of the present invention, there is provided a method of forming a fabric sensor for measuring body movement comprising forming first and second gripping sections and a third sensing section intermediate of the first and second sections, the third sensing section having a lower amount of grip than either the first or second gripping sections.

According to a third aspect of the present invention, there is provided a garment comprising one or more substantially tubular sections for receiving a body portion and including one or more fabric sensors according to the first aspect of the invention.

According to a fourth aspect of the present invention, there is provided a method of forming a garment comprising forming one or more substantially tubular sections for receiving a body portion and including one or more fabric sensors according to the first aspect of the invention.

Owing to the invention, it is possible to provide a fabric sensor, and a garment incorporating the sensor, that will be held in place and sense body movement, but will not give false readings caused by inadvertent moving of the sensor. By having a gripping section on either side of the sensing section, the sensing section is held in place, but the areas of lower grip have sufficient flexibility to allow them to absorb the movement of other parts of the body.

Advantageously the first and second gripping sections have substantially the same amount of grip as each other. This simplifies the manufacture of the fabric sensor. Preferably, the third sensing section has approximately zero grip.

In a preferred embodiment, the third sensing section comprises a construction of first and second yarns, the first yarn being of a conductive nature, and the second yarn being of an elastic nature. The construction of the third sensing section consists of a knit of the first and second yarns. This construction allows the sensing section to be easily and cheaply manufactured, while providing a good sensor. The resistance of the sensing section will vary in proportion to the amount of stretch that occurs, thereby providing an efficient measure of the amount of bend of the particular joint being measured. The construction of this sensor is described in detail in Bickerton, M. “Effects of fibre interactions on conductivity within a knitted fabric stretch sensor”, proceedings of IEE Eurowearables '03 pp. 67-72, 4th-5th September 03, ISBN 0 85296 282 7, ISSN 0537-9989.

Preferably, at least one of the first and second gripping sections has an amount of grip that varies over its width. By having a varying grip, a more comfortable fabric sensor is provided for the user.

Advantageously, each of the first and second gripping sections and the third sensing section encircle a body portion. This ensures a good grip on the user's body portion without being too uncomfortable.

Ideally the first and second gripping sections and the third sensing section are formed of single piece of fabric. The garment itself is also, ideally, formed from a single piece of fabric. The manufacturing process is therefore simplified.

Embodiments of the present invention will now be described, by way of example only, with reference to the accompanying drawings, in which:

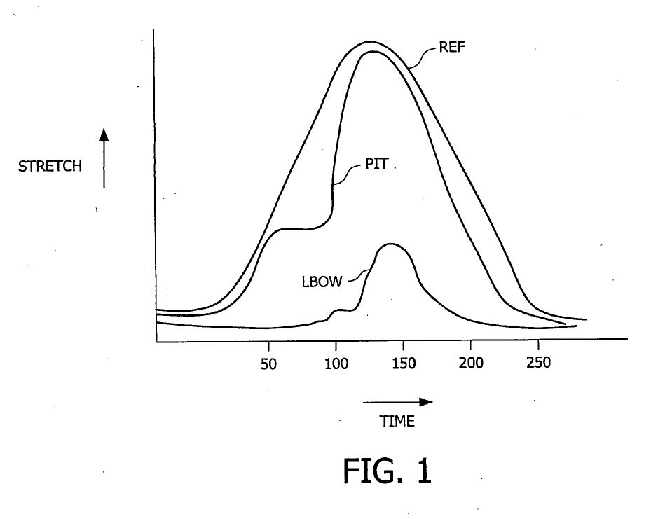

FIG. 1 is a graph showing stretch sensor values in a prior art sensor system,

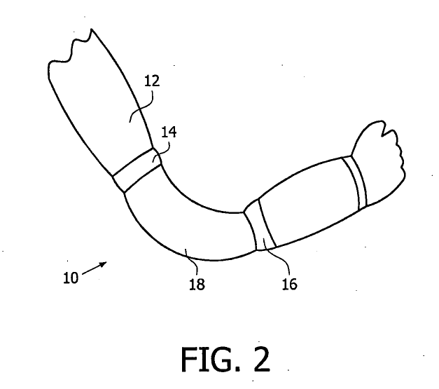

FIG. 2 is a perspective view of a fabric sensor,

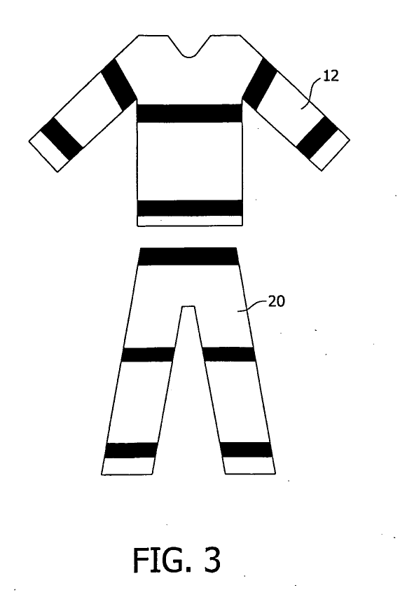

FIG. 3 is a front view of two garments incorporating several sensors of the type shown in FIG. 2,

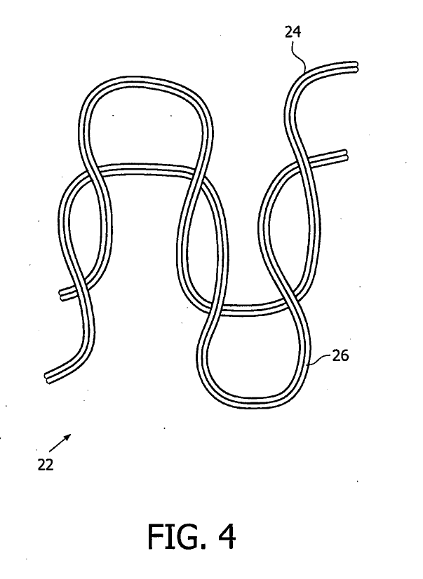

FIG. 4 is a perspective view of first and second yarns, and

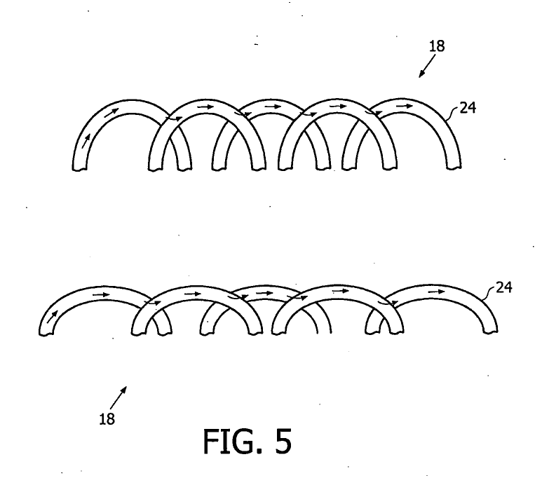

FIG. 5 is a simplified perspective view of a section of the yarn of FIG. 4 showing the change in the yarn when stretched.

FIG. 1 shows a graph showing stretch sensor values in a prior art sensor system, which illustrates the problem of the prior art systems. This graph shows the values returned by two different fabric sensors on a garment, and a reference goniometer, as a user raises their arm from a vertical position at their side, to a horizontal position, with their arm out from the side of their body, and back down again. The lines on the graph are marked REF for the reference goniometer, LBOW for a stretch sensor on the back of the elbow, and PIT for a stretch sensor on the inside of the armpit.

The reference reading REF shows the user raising their arm to the horizontal, at time 125, and then back down again at time 250. The reference REF is the reading that would be expected at the armpit. As can be seen from the graph of the armpit sensor PIT, at time 50, an artefact occurs, which is caused by the sleeve of the garment slipping. Likewise, the elbow sensor LBOW, which should remain constant throughout, shows a certain amount of movement at time 125. This again is an unwanted artefact. The effective length of the arm increases as it is raised and if held tight around the wrist, the arm begins to stretch, which pulls the elbow sensor, giving a false reading.

FIG. 2 shows the improved fabric sensor 10, in the elbow region of a garment 12. The fabric sensor 10 is for measuring body movement and comprises first and second gripping sections 14 and 16, and a third sensing section 18, which is intermediate of the first and second sections 14 and 16.

The third sensing section 18 has a lower amount of grip than either the first or second gripping sections 14 and 16, with the first and second gripping sections 14 and 16 having substantially the same amount of grip as each other. In this embodiment, the third sensing section has, in fact, zero grip. The two gripping sections 14 and 16 are made of a smaller diameter than the sensing section 18. Alternatively, they can include within the fabric a certain amount of elasticated material to achieve the gripping effect.

The gripping provided by the sections 14 and 16 ensures that the sensing section remains isolated from the rest of the garment and in the correct position relative to the body portion for which it is taking a reading. On the non-sensing section sides of the first and second gripping sections 14 and 16 is non-elasticated material, which allows a certain amount of give in the garment to provide the flexibility to avoid the fabric sensor being pulled out of position.

Each of the first and second gripping sections 14 and 16 and the third sensing section 18 encircle the body portion (in this example the arm) of the user, thereby ensuring a stable sensor arrangement. The first and second gripping sections 14 and 16 and the third sensing section 18 are formed of single piece of fabric. The gripping of the sections 14 and 16 is achieved by those sections being of a smaller diameter than the sections on either side of them. This ensures that they are in closer contact with the user's arm than, for example, the sensing section 18. The gripping sections 14 and 16 are tighter and not laterally stretchy when compared to the stretching section 18, in order that any stretching that occurs, will occur at the sensing section 18.

In a further embodiment, at least one of the first and second gripping sections 14 and 16 has an amount of grip that varies over its width. Preferably both sections 14 and 16 have this grading of grip level over their width. This provides a more comfortable fit for the user.

FIG. 3 show the garment 12, which is for the upper body of the user and also illustrates a second garment 20, which is for the lower part of the body. The thick black lines illustrate schematically the gripping sections in the fabric sensor, with the sensing sections (not shown) lying in between each pair of gripping sections. A gripping section can have a sensing section on both sides of it, the gripping section effectively acting as a locating section for two different sensing sections. Both garments 12 and 20 comprise a plurality of substantially tubular sections for receiving a body portions and including several fabric sensors for measuring body movement. Each garment 12 and 20 is formed from a single piece of fabric.

FIG. 4 shows a close up of a small portion of the third sensing section 18. This section 18 comprises a construction 22 of a first yarn 24 and a second yarn 26, the first yarn 24 being of a conductive nature, and the second yarn being of an elastic nature 26. The construction 22 of the third sensing section 18 consists of a knit of the first and second yarns 24 and 26. The first yarn 24 is preferably of a resistive nature such as carbon, and the second yarn 26 is of an elastic nature, such as elastic, or Lycra.

Using the basic structure illustrated in FIG. 4, it is possible to construct a knitted fabric sensing section 18 that increases or decreases resistance when stretched.

Elongation of the knitted fabric sensing section 18 causes an increase in measured resistance due to an increase in the length of the conduction paths through the fabric. The sensing section 18 is connected electrically in such a way that the current flows along the length of the fabric, perpendicular to the direction of the carbon strands. Conduction therefore occurs via inter fibre contact.

FIG. 5 shows a simplified view of a cross section through the knitted stretch sensing section 18. In the upper part of the Figure, an unstretched portion of the section 18 is illustrated. Current can be seen to flow along the length of the fabric strip, as indicated by the arrows, through the carbon fibres 24, passing between each, at the point of contact. As the fabric is stretched, shown in the lower part of the Figure, elongation of the loops will increase the total length of carbon fibre that the current must pass along, and so increase the measured resistance of the sensing section 18.

In fact, the operation of the sensing section 18 is more complicated than as shown in FIG. 5. Whilst it might be imagined that the change shown in FIG. 5 would give a 2 or 3 times increase in sensor resistance when stretched, in fact a sensing section 18 constructed according to the design shown in FIG. 4 shows a factor of 5 to 10 in the observed change in resistance of the sensing section 18.

Other factors that affect the resistance of the sensing section 18, when it is stretched, include the making and breaking of inter-fibre contacts. Conduction does occur along the length of the sensor by inter-fibre contact. Although the fibres of carbon 24 in the knit structure travel perpendicular to this direction, and current is free to flow along these fibres; due to the very high resistance of the carbon strands, inter fibre contact along this plane will also have a significant effect on the overall resistance of the fabric, by greatly reducing the resistance between inter-row connections. These connections do not occur at every interconnecting loop because the elastic fibres 26, intertwined with the carbon fibres 24, keep many of the inter-row carbon loops separated from each other. The making and breaking of inter fibre contacts also occurs between adjoining rows of the carbon knit. The carbon loops are inevitably of a varying size, and some of these will be touching each other, allowing current to flow between rows. If a shorter loop were touching a longer loop which lay over it, then as the fabric stretches the lower loop, being shorter, will flatten at a faster rate than the upper loop and so lose contact, whereas were the situation reversed, with the lower loop being the longer, then when stretched, contact would remain. Therefore the net result of stretching the fabric will be a reduction of inter-row fibre contacts, and therefore an increase in overall resistance.

The garment 12 incorporating the fabric sensor 10 has a number of application areas, which include:
Sports coaching. By sensing the dynamic body position it is possible to analyse the swing or movement of a person engaged in sports and give feedback to improve their performance.
Physiotherapy: By wearing an appropriate sensing garment, for example tights, a person can perform exercises in their own home and the system can record these and again coach them to ensure they are doing them correctly. This will help hospital physiotherapy department become more efficient.
Gaming: A sensor jacket could provide a means of interface to a video game. For example, the user could do large movements in a fighting game.

1. A fabric sensor (10) for measuring body movement comprising first and second gripping sections (14, 16) and a third sensing section (18) intermediate of the first and second sections (14, 16), the third sensing section (18) having a lower amount of grip than either the first or second gripping sections (14, 16).

2. A fabric sensor according to claim 1, wherein the first and second gripping sections (14, 16) have substantially the same amount of grip as each other.

3. A fabric sensor according to claim 1, wherein the third sensing section (18) has approximately zero grip.

4. A fabric sensor according to claim 1, wherein the third sensing section (18) comprises a construction (22) of first and second yarns (24, 26), the first yarn (24) being of a conductive nature, and the second yarn (26) being of an elastic nature.

5. A fabric sensor according to claim 4, wherein the construction (22) of the third sensing section (18) consists of a knit of the first and second yarns (24, 26).

6. A fabric sensor according to claim 1, wherein at least one of the first and second gripping sections (14, 16) has an amount of grip that varies over its width.

7. A fabric sensor according to claim 1, wherein each of the first and second gripping sections (14, 16) and the third sensing section (18) encircle a body portion.

8. A fabric sensor according to claim 1, wherein the first and second gripping sections (14, 16) and the third sensing section (18) are formed of single piece of fabric.

9. A method of forming a fabric sensor for measuring body movement comprising forming first and second gripping sections (14, 16) and a third sensing section (18) intermediate of the first and second sections (14, 16), the third sensing section (18) having a lower amount of grip than either the first or second gripping sections (14, 16).

10. A method according to claim 9, wherein the first and second gripping sections (14, 16) have substantially the same amount of grip as each other.

11. A method according to claim 9, wherein the third sensing section (18) has approximately zero grip.

12. A method according to claim 9, wherein the third sensing section (18) comprises a construction (22) of first and second yarns (24, 26), the first yarn (24) being of a conductive nature, and the second yarn (26) being of an elastic nature.

13. A method according to claim 12, wherein the construction (22) of the third sensing section (18) consists of a knit of the first and second yarns (24, 26).

14. A method according to claim 9, wherein at least one of the first and second gripping sections (14, 16) has an amount of grip that varies over its width.

15. A method according to claim 9, wherein each of the first and second gripping sections (14, 16) and the third sensing section (18) encircle a body portion.

16. A method according to claim 9, wherein the first and second gripping sections (14, 16) and the third sensing section (18) are formed of single piece of fabric.

17. A garment (12; 20) comprising one or more substantially tubular sections for receiving a body portion and including one or more fabric sensors (10) according to claim 1.

18. A garment according to claim 17, wherein the garment (12; 20) is formed of a single piece of fabric.

19. A method of forming a garment (12; 20) comprising forming one or more substantially tubular sections for receiving a body portion and including one or more fabric sensors (10) according to claim 1.

20. A method according to claim 19, wherein the garment (12; 20) is formed of a single piece of fabric.
